(19) United States
(12) Patent Application Publication
(10) Pub. No.: US 2011/0313235 A1
(43) Pub. Date: Dec. 22, 2011

(54) APPARATUS FOR MODULATING PERFUSION IN THE MICROCIRCULATION OF THE BLOOD

(75) Inventor: Peter Gleim, Liechtenstein (DE)

(73) Assignee: Peter Gleim, Triesen (LI)

(21) Appl. No.: 13/145,963

(22) PCT Filed: Jan. 26, 2010
(86) PCT No.: PCT/EP10/50852

§ 371 Date: Aug. 16, 2011

(30) Foreign Application Priority Data
Jan. 29, 2009 (EP) 09151615.3

(51) Int. Cl. A61N 2/02

(52) U.S. Cl. 600/14

(57) ABSTRACT

The invention relates to an apparatus for modulating perfusion in the microcirculation of the blood. The apparatus consists of a first device for generating a pulsed electromagnetic field having a synchronous or asynchronous specific pulse sequence of a periodic electromagnetic field, including a first group of pulses with a pulse time of 0.1 to 0.2 seconds at 35 to 100 μT and a second group of pulses of 10 to 30 seconds at 2 to 40 μT, a second device for circulating the lymph flow, said second device being used to trigger massaging pulses at increasing and decreasing pressures via several massaging arrangements arranged next to each other, and a third device for emitting infrared radiation, said third device emitting at least one heat pulse, as well as a control unit by means of which the pulses of the three devices are controlled such that two or three devices simultaneously emit pulses. Significant improvements of individual parameters of the microcirculation and also the macrocirculation of the blood are achieved with the apparatus.

The invention relates to an apparatus for modulating the perfusion in the microcirculation of the blood.

It is already known to influence microcirculation by electromagnetic pulses.

From EP 0 995 463 a device is known by means of which biological processes in the human body are influenced by pulsed electromagnetic fields, particularly in order to increase O₂ depletion and to stimulate metabolic processes. The single pulses can be in accordance with a function represented by a formula.

WO 2008/025731 describes a device for generating a pulsed electromagnetic field with periodic pulses having increasing and decreasing envelope curves as a function of particular measured data of the microcirculation of the blood.

The object of the invention is to provide an apparatus which is suitable for the purpose of significantly increasing the microcirculation of the blood.

An apparatus for modulating perfusion in the microcirculation of the blood according to the invention consists of a first device for generating a pulsed electromagnetic field, comprising at least one pulse generator and a magnetic coil, having a pulse sequence of at least two synchronous or asynchronous groups of pulses, wherein for the first group of pulses the pulse time is 0.1 to 0.2 seconds at a magnetic flux density of 35 to 100 μT and for the second group of pulses the pulse time is 10 to 30 seconds at a magnetic flux density of 2 to 40 μT; a second device for enhancing the lymph flow, said second device being used to trigger massaging pulses via a massaging arrangement, wherein the massaging arrangement comprises several circuits of compression chambers, the compression chambers being arranged horizontally next to each other and the circuits being arranged above each other, said circuits being configured such that, via a pulsing device and one or more pumps connected thereto, gradient increasing pressure pulses can be triggered for the compression chambers in one half of the circuit and gradient decreasing pressure pulses can be triggered for the chambers in the other half of the circuit; a third device for emitting infrared radiation, said third device emitting at least one heat pulse and having an infrared spectrum of 80 to 100% infrared-A, 0 to 19% infrared-B and 0 to 1% infrared-C; a control unit by means of which the pulses of the three devices are controlled such that either the first and the second device simultaneously emit pulses or the first and the third device simultaneously emit pulses or the first, second and third device simultaneously emit pulses.

The combination of two or three devices within the inventive apparatus for modulating perfusion leads to effects with regard to the microcirculation of the blood which are clearly more pronounced than the effects which can be achieved with each individual device on its own. Details of this synergistic effect will be explained further below.

The functional state of an organ is substantially determined by the functional state of its microcirculation. Today, it is generally accepted that most functional disorders or pathological conditions of organs are at least determined in their course, if not even triggered, by microcirculatory disorders. Microcirculatory disorders often arise out of macrocirculatory disorders and may gradually develop their own dynamics, which regardless of the macrocirculatory processes vitally affect the course of the disease or even dominate it. An enhancement of the functional activities, healing or restitution processes in the organs are not possible without microcirculation, i.e. the transport processes in the area of the microvessels, being involved. If anything, the symptoms of a disorder or a disease can be influenced to a small extent and temporarily at best if microcirculation is restricted. Influencing microcirculation is therefore of particular importance.

In the apparatus according to the invention, the first device for generating a pulsed electromagnetic field consists of at least one pulse generator and at least one coil connected thereto. Advantageously, the coil is a flat structure, preferably a mat, for generating the electromagnetic field. This structure comprises several coils which are circularly or rectangularly arranged next to each other or in partial overlap, via which coils the electromagnetic pulses or groups of pulses are passed on to a skin surface of a user, said skin surface being in contact therewith at a small distance.

The coil can also be arranged in a hand-held intensive applicator and thus cover a relatively small area of 20 to 150 cm² to permit localized applications.

In the invention, “synchronous group of pulses” means a continuous base signal of the second group of pulses of e.g. 2 to 40 μT and a higher supplementary signal of 35 to 100 μT of the first group of pulses superimposed thereon at an interval of 10 to 30 seconds, preferably 10 to 20 seconds, the pulse time of the supplementary signal being only 0.1 to 0.2 seconds. In any case the supplementary signal is at least 10 μT and at most 90 μT above the base signal.

“Asynchronous group of pulses” means a base signal of the second group of pulses which is aborted after 10 to 30 seconds, an immediately following supplementary signal of 0.1 to 0.2 seconds of the first group of pulses, and an immediately following base signal again, followed by a supplementary signal again, and repetitions of these sequences.

The pulse sequence of the first device consists of groups of pulses with an associated pulse time and an associated magnetic flux density.

For the invention the pulse time of a first group of pulses is preferably 0.1 to 0.2 seconds at a magnetic flux density of 40 to 90 μT. This first group of pulses preferably occurs in alternation with a second group of pulses. The pulse time of the second group of pulses is preferably 10 to 20 seconds, particularly 15 to 20 seconds. The magnetic flux density of the second group of pulses is preferably 5 to 34 μT. It is particularly preferred that the first group of pulses occurs two to six times per minute, in particular two to four times, particularly preferably two to three times.

It is particularly preferred if the magnetic flux density of the first group of pulses is 30 to 80 μT higher than that of the second group of pulses, in particular 35 to 65 μT higher.

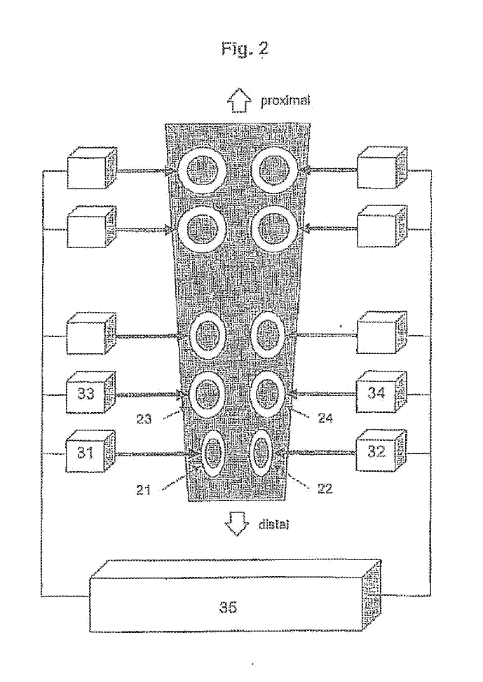

The pulse sequences consist of single pulses, the amplitudes of which follow e.g. an exponential function. A particularly preferred exponential function is described in EP 995463 B1, with y=x³·e^{sin(x}^{³}⁾, wherein the formula expresses the progression of the amplitude y over the time x. The single pulses then have a progression as shown, for example, in FIG. 2 of EP 995463 B1.

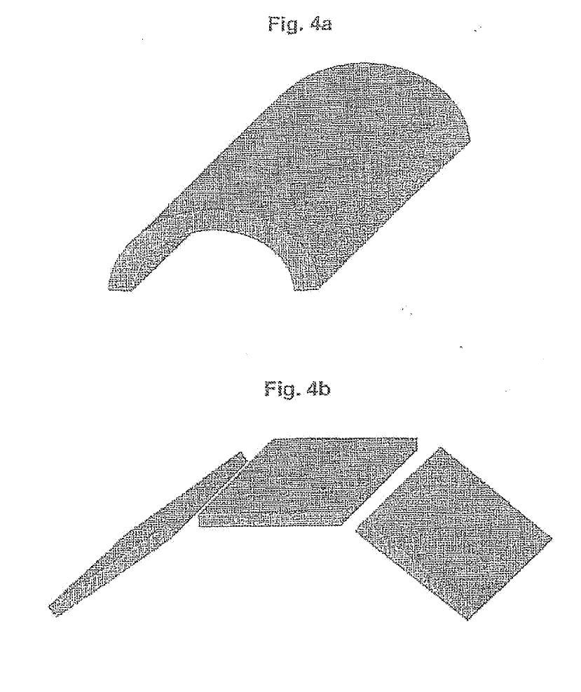

The single pulses can also have non-exponential progressions, representing increasing and decreasing envelope curves with harmonic or inharmonic resonant curves as in WO 2008/025731. Alternating groups of pulses with such resonant curves are presented e.g. in FIGS. 4c to 4f of WO 2008/025731. Groups of pulses with single pulses, the amplitude of which corresponds to an exponential function, are preferred for the present invention.

In a particularly preferred embodiment of the invention, the second group of pulses with approximately 9 to 22 μT occurs over a time of approximately 15 to 25 seconds, and, in alternation with this, supplementary pulses occur for 0.1 to 0.2 seconds of a group of pulses of 30 to 45 μT, and this alternating sequence is emitted by the first devices for a total period of 4 to 20 minutes. The pulses are emitted by a pulse generator with a coil arrangement in the form of a mat connected thereto and are controlled via the control device. The person to be treated is lying on the mat at a small distance of 5 to 20 mm from the coil arrangements.

If the first device for generating a pulsed electromagnetic field is present in the form of a hand-held intensive applicator as described above, the pulses of the second group of pulses can be twice or three times as high and thus be at 6 to 70 μT, in particular 30 to 68 μT, while the supplementary pulses of the first group of pulses are 60 to 95 μT, in particular 80 to 95 μT, and the latter exceed the ones of the second group of pulses with a difference of at least 10 μT.

The coil arrangement can be realized in such a manner that one or more flat coils, which can be circular or rectangular, are distributed in a flat element, such as a mat, next to each other or in partial overlap. Such a mat or cuff is then brought into contact with a part of the human body. The coil arrangement can also be configured as a small-surface hand-held or intensive applicator which is guided across the surface of the skin.

The frequency is preferably within the range from 8 to 40 Hz.

The second device relates to circulating the lymph flow. Microcirculation with its transcapillary fluid exchange in the capillary flow region is closely connected to the initial lymph flow. In contrast to the transport of the plasma/blood cell mixture in the blood vessels, the lymph flow is a “one-way street”. Comparatively low pressure gradients are required for moving the extravascular fluid in the intercellular space to the lymph capillaries and for its penetration through the lymphatic endothelium into the lumen of the blind-ended lymph capillaries (initial lymph flow) as well as for transporting the lymph on in the larger lymph flow tracts. By a suitable effleurage of the skin a higher pressure gradient can therefore be built up in the tissue mechanically, said pressure gradient resulting in an acceleration of the lymph flow (lymphatic drainage).

Aside from manual lymphatic drainage, which has been practiced successfully for quite some time now and which requires the user to have specially trained skills, also several semi-automatic or automatic treatment appliances are in use for lymphatic drainage (limb cuffs with pressure chambers, etc.). The effects produced by these commercially available appliances are not satisfactory, however. According to the invention, however, the stimulation of the lymph flow can be significantly increased.

The basic principle of the technical realization of this new treatment device (limb cuffs with a system of particular regulatable pressure chambers) is based on the accepted state of knowledge in manual lymphatic drainage. Successive locally circumscribed circular effleurage strokes of a pressure alternating in a defined manner are applied to the lower limbs from the distal to the proximal position at a specific rhythm, taking into account the efflux direction of the lymph flow.

A preferred second device of the invention therefore consists in massaging arrangements in a cuff adapted to the body, e.g. for a leg. The massaging arrangements are e.g. individual compression chambers, which are arranged horizontally next to each other and vertically (from bottom to top) above each other in circular planes. The chambers are connected to each other in series and are actuated by compressed air or a fluid/pressure system.

In the compression chambers which are circularly arranged next to each other horizontally, in one half of the circuit the chambers next to each are successively subjected to increasing pressure pulses, and then in the other half of the circuit the subsequent chambers next to each other are subjected to increasingly fewer pressure pulses, such that the pressure in the circuit first increases and then decreases again.

With the cuff closed, the chambers arranged next to each other form a circuit, e.g. a circuit of 7 chambers. The pressure can be built up via a pulsing device and one or more pumps connected thereto so as to increase from chamber 1 to chamber 7, or it can be built up in the one half of the circuit from chamber 1 to chamber 4 (boost phase) and be reduced again from chamber 5 to 7 (relaxation phase), i.e. the chambers are driven via a control device one after the other at different pressures. This first circuit is located at the end of the cuff at the height of the ankle. The next circuit above it in the direction of the knee/thigh again has several chambers located next to each other. Further circuits in the cuff then follow, e.g. until the end of the thigh. Pressure actuation is performed successively from the first circuit to the last circuit from the distal to the proximal position, and always within the corresponding circuit with a gradient boost and relaxation phase, the pressure for each massaging circuit being able to be controlled individually to be more or less intense. As a rule, the pressure in the massaging circuits is increased successively from one circuit plane to the next from the distal to the proximal position.

Advantageously, each circuit is associated with one pump. The same pumps can then also be responsible for the action phase in the next circuit. However, it is also possible to provide a dedicated pump for each chamber which can then be controlled via the pulsing device.

The function of the second device for circulating the lymph flow is applied over a period of 5 to 20 minutes. The force applied via the pressure chambers generally corresponds to the force which is applied during manual lymphatic drainage. The force is within the range from 2 to 65 N, in relation to the surface of the skin.

Preferably, the force in the pressure chambers which come into contact with the lower limbs is 2 to 35 N in the relaxation phase and 10 to 65 N in the boost phase. For the chambers which come into contact with the upper limbs the force is preferably 2 to 20 N in the relaxation phase and 5 to 35 N in the boost phase. This is in relation to a skin area of about 18×18 cm in each case. The chambers are to be designed in accordance with the forces applied.

It is particularly advantageous if the rhythm for the pressure build-up achieved by pumps is adapted to the rhythm of vasomotion. Therefore, 2 to 4 boost/relaxation phases are carried out per minute.

Controlling the pressures in the form of boost and relaxation phases in the sequence of the chambers within one circuit plane and the design of different pressures (force) in the upper and the lower region of the limbs is an entirely new principle of operation and is far superior to manual lymphatic drainage and to appliances for instrumental intermittent compression (German: apparative intermittierende Kompression, AIK) available on the market so far.

The invention therefore also relates to a massaging device for circulating the lymph flow, characterized in that in a flat structure, massaging arrangements such as 21; 22; 23; 24 are arranged horizontally next to each other and vertically in several circular planes above each other, where a pulsing device and a pump controller 35 and more pumps such as 31, 33 connected thereto are used to trigger gradient increasing pressure pulses for the chambers 21; 23 in one half of the circuit, and the pumps such as 32; 34 are used to trigger gradient decreasing pressure pulses for the chambers 22; 24 in the other half of the circuit.

Advantageously, the function of the second device as well as of the first and third devices is applied as a function of the microcirculation criteria presented in the following.

Evaluation of the stimulation of the (initial) lymph flow is done based on accepted criteria for characterizing the microcirculation of the blood, such as the number of blood cell perfused nodal points in the defined microvascular network (nNP), changes in the venular flow rate (ΔQven), venular oxygen depletion (ΔpO₂), as well as based on the flow rate of the initial lymph (ΔQ_{L}).

The oxygen depletion of the venule side ΔpO₂ is given as the percentage of change as compared to the corresponding initial value at the time t=0. The absolute difference is determined between the oxygen saturation of haemoglobin in the afferent arterioles and the efferent venules in the network of a selected tissue target. As the target, sections of the skin or the intestine tissue are selected, which contain the desired blood vessel networks of the organism and furthermore belong to the immunologically active organs. In addition, they are readily accessible for non-invasive measurements.

For the number of the currently blood cell perfused nodal points in the defined microvascualar network, nNP, the number of blood cell perfused branching sites within this network is used as a measure of the state of distribution of the blood. v_{RBC}=80 μm/s is defined as the limit flow velocity of the red blood cells. Evaluation is done using + or − (compared to the defined initial value n=60). Borderline cases are scored as +0.5 or −0.5.

The venular flow Qven and the arterial flow Qart constitute the particle flow (blood cell flow) in defined venules or aterioles, respectively. They are defined in μm³/s.

With a lymphatic drainage according to the invention carried out for 15 minutes, for example, a 15% increase in nNP is observed after about 20 minutes, which then slowly decreases again. ΔpO₂ and ΔQven increase to about 10% within 10 minutes and then decrease again.

When combining the first device for generating a pulsed electromagnetic field with the second device for circulating the lymph flow, a comparison of these characteristics of the microcirculation of the blood shows no additive effect, but very distinctly a significant synergism.

For nNP, the percentage of increase achieved with the first device on its own is about 1%, with the second device on its own about 15%, and with both combined about 22%.

For ΔQven, the percentage of increase achieved with the first device on its own is about 5%, with the second device on its own about 10%, and with both combined about 18%.

For Δp0₂, the percentage of increase achieved with the first device on its own is about 2.5%, with the second device on its own about 11%, and with both combined about 20%.

For ΔQ_{L}, the percentage of increase achieved with the first device on its own is about 9%, with the second device on its own about 10%, and with both combined about 31%.

It will be appreciated that the combined application of a pulsed electromagnetic alternating field and an effective lymphatic drainage effects a distinct increase in the initial lymph flow. Moreover, greater changes in the characteristics of the microhaemodynamic functional characteristics occur.

Furthermore, it has been found that also macrocirculatory changes appear which indicate a physiologically favorable influence on the venous backflow into the right atrium.

The third device in the context of the apparatus according to the invention relates to a device for emitting infrared radiation.

What is referred to as heat radiation in the strict sense is that part of the spectrum of electromagnetic waves which is outside the range of visible light in the longer-wavelength infrared. Electromagnetic waves of wavelengths of λ>780 nanometers are referred to as infrared rays.

The nature of the emission of thermal radiation lies in a conversion of heat energy into radiant energy. The wavelengths of the thermal radiation of a solid body form a continuous spectrum (e.g. solar spectrum). The focus of emission for low temperatures is in the range of large wavelengths (infrared), for higher temperatures in the range of shorter wavelengths.

With regard to their different depths of penetration into the human skin, three subareas of infrared radiation are to be distinguished: infrared-A, wavelength 800 to 1400 nm, depth of penetration into the skin up to 6 mm; infrared-B, wavelength 1400 to 3000 nm, depth of penetration into the skin up to 2 mm; infrared C, wavelength 3000 to 10 000 nm, depth of penetration into the skin up to 1 mm.

Three processes occur when radiation impinges on the skin:
absorption, where part of the incident radiation enters the tissue and is absorbed;
refraction, where part of the incident radiation is refracted at interfaces according to the law of refraction;
reflection, where part of the incident radiation is reflected by the surface according to the law of reflection.

It is a widespread error to assume that a defined transfer of radiant energy into the skin is a very simple process which is easy to understand. Both the technical realization of suitable treatment appliances and the specification of effective treatment measures which have no side effects or only few side effects require profound scientific knowledge of physical principles and sufficient knowledge of the effects of infrared radiation on the structures of the skin tissues, including their regulative mechanisms.

More research is still needed with regard to the mechanisms acting when heat radiation enters the skin. This applies to the local regulation mechanisms of microcirculation in the skin, aspects of the temperature receptors, the course of biochemical processes, including the temperature optima of enzymatic processes, etc.

With regard to the course of biochemical reactions, a relationship between temperature and metabolic activity has long been known as “van't Hoff's rule”. According to this rule, the velocity of biochemical reactions (metabolic intensity) increases 2- to 3-fold for an increase in temperature by 10° C. The diversely linked metabolic processes occurring under heat supply have not been sufficiently researched in detail yet, however.

What has been largely explained, on the other hand, are the mechanisms of temperature regulation acting when heat is emitted via the skin (convection of heat energy in the blood stream, heat conduction through the tissues to the skin surface, radiation of heat energy via the skin surface, emission of evaporation heat while sweating; shifts in the tissue masses of the core of the body and the body surface, central nervous influences, etc.).

It has been found that the following changes in characteristics occur in the microcirculation of the skin under heat radiation:
(1) increased influx of blood from the networks of the deeper tissues and redistribution of the blood volume in the skin tissue between the horizontal microvessel networks in the skin tissue via vertical connections
(2) arteriole diameter increases
(3) arteriolar-venular pressure gradient increases
(4) venular efflux increases
(5) number of blood cell perfused capillaries increases.

The consequences of these changes in the skin tissue are:
distribution of the flowing blood volume over more capillaries (capillaries which have previously been predominantly plasma perfused now are predominantly blood cell perfused)
shortening of the diffusion paths for the transcapillary oxygen exchange
venular increase in oxygen depletion
disaggregation of red blood cells as a result of increased flow velocities
improvement of the flow properties of the blood in the skin tissue
more favorable microhaemodynamic boundary conditions for an unhindered progression of the first steps of an immune reaction (influx and distribution of white blood cells in the microvascular networks of the skin, rolling phenomena and adhesions to the endothelium, transmigrations into the tissue)
a broader amplitude of adaptation of the microcirculation of the skin depends on the type of infrared radiation (IR-A, IR-B or IR-C or combination of the partial IR radiations), on the radiation intensity Iₑ, on the duration of irradiation, and dependence on the condition of the treated skin and the organism as a whole.

A suitable third device for emitting infrared radiation is one which emits heat pulses, the spectrum of which corresponds to natural sunlight after it has passed the atmosphere of the earth. This infrared spectrum consists of about 80% infrared-A, 19% infrared-B and approximately 1% infrared-C. Industrial surface radiators with a proportion of only about 5% infrared-A are not suitable.

A third device is particularly preferred, the spectrum of which consists substantially of 90% infrared-A, 9% infrared-B and 1% infrared-C, in particular of 100% infrared-A. A preferred wavelength is 940 nm.

The energy input to the skin tissue has to occur homogeneously to avoid undesirable interactions of the closely interlinked microvessels in the skin tissue and thus local “irritations” of the heat receptors in the skin.

The mode of operation of the third device is, therefore, strongly influenced by the ambient conditions, in particular the site temperature, which should generally be within the range from 18 to 25° C. to create a comfortable temperature for the patient. What is to be effected in the irradiated region of the skin is, to a biologically relevant extent, a stimulation of the microcirculation by an increased venular efflux with simultaneously enhanced distribution of the blood in the capillary networks. This can only be achieved if the interaction of local and neural controls of the skin perfusion can be influenced in a physiologically favorable manner.

For this purpose the third device is required to emit a heat radiation pulse at a distance of 0.15 to 0.40 m from the irradiated skin tissue, said heat radiation pulse achieving an increase in the mean temperature of the irradiated skin field by 2 to 5° C. at a radiation pulse duration of 10 to 30 minutes. This can be done using e.g. a hand-held applicator with a radiating surface of 50×50 mm, which is equipped with about 40 infrared-A diodes and which achieves a radiated power of 0.6 W.

With a power of 40 mW per diode at 20 mA, a luminance of 3500 millicandelas (mcd) per diode is achieved.

Also, a surface applicator having a radiating surface of 109×270 mm can be used which has about the same density of infrared-A diodes as the hand-held applicator and which achieves a radiated power of 5.3 W. Several surface applicators can be combined with each other.

To achieve a homogeneous energy input to the skin tissue, the distances from the skin surface are to be taken into account in accordance with the theorem of intersecting lines. As the energy per area decreases with the square of the distance from the radiation source, inhomogeneous fields can form in the case of skin surfaces with small radii of curvature.

It is therefore advantageous to have a semicircularly bent realization of the third device, with a length approximately corresponding to the length of the human body (100 to 200 cm).

Another embodiment relates to plate-shaped third devices, where one plate is arranged horizontally and e.g. two plates are arranged on the left and right sides next to the horizontal plate, inclined at an angle of 30 to 60° to the tissue area to be irradiated.

With such an exemplary embodiment, 120 to 200 diodes can be arranged per plate (luminous surface), preferably 150 to 170 diodes, such that the three pivotable plates in total have preferably 450 to 510 diodes.

Modulation of the heat pulse in the form of the luminance in candela (cd) is preferably done with a digitally processed signal of the first device for generating a pulsed electromagnetic field, and thus according to the pulse sequences as in this first device. This may not be obvious, but it is, in this way, adapted to the biorhythm.

When combining the first device for generating a pulsed electromagnetic field with the third device for emitting infrared radiation, a comparison of the characteristics of the microcirculation of the blood shows no additive effect, but likewise a significant synergism, as has been the case for the combination of the first with the second device.

For nNP, the percentage of increase achieved with the first device on its own with a different group of probands is about 7%, with the third device on its own about 14%, and with both combined about 25%.

For ΔQven, the percentage of increase achieved with the first device on its own is about 5%, with the third device on its own about 13%, and with both combined about 22%.

For ΔpO₂, the percentage of increase achieved with the first device on its own is about 5%, with the third device on its own about 12%, and with both combined about 28%.

The combined application of the first and the third device thus leads to a clearly improved and synergistic effect with regard to specific characteristics of microcirculation.

A further improvement as to the effects is achieved, to an extent which was not to be expected, by combining the first device for generating a pulsed electromagnetic field with the second device for circulating the lymph flow and the third device for emitting infrared radiation in an apparatus for modulating perfusion of the microcirculation of the blood.

When combining the first and the third device, ΔpO2 is at about 28%, as explained above. The second device on its own leads to an improvement of about 3%. All three devices combined result in about 37% for ΔpO₂.

Moreover, the tests, in particular those which have been carried out with a combination of the first and the second device and of the first, second and third device, show that also macrocirculatory changes can be achieved which indicate a physiologically favorable influence on the venous backflow into the right atrium. The pressure difference between the right atrium and the vena cava is about 5% for one group of probands using the combination, while the sum of the individual devices remains below 4%.

In the massaging device according to the invention the flat structure is preferably a leg cuff having compression chambers as massaging arrangements. Further advantageous embodiments are described with reference to the second device mentioned above.

The invention will now be explained in greater detail by means of examples and with reference to the attached drawing, in which:

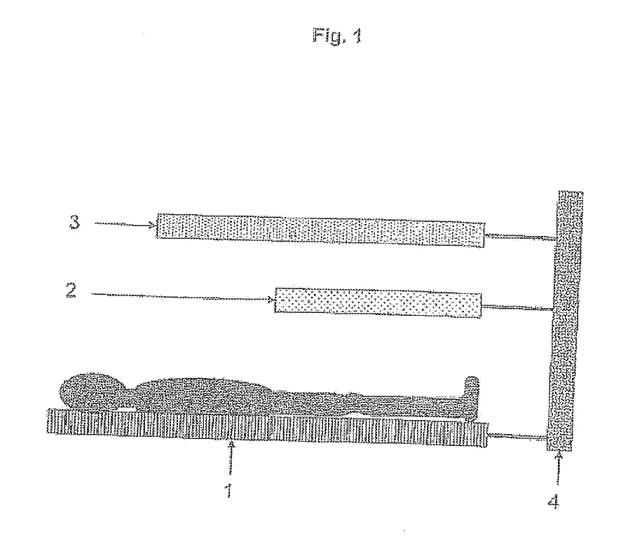

FIG. 1 shows a schematic representation of the regions of application of the apparatus on human beings

FIG. 2 shows a schematic representation of a device for circulating the lymph flow in the form of a cuff

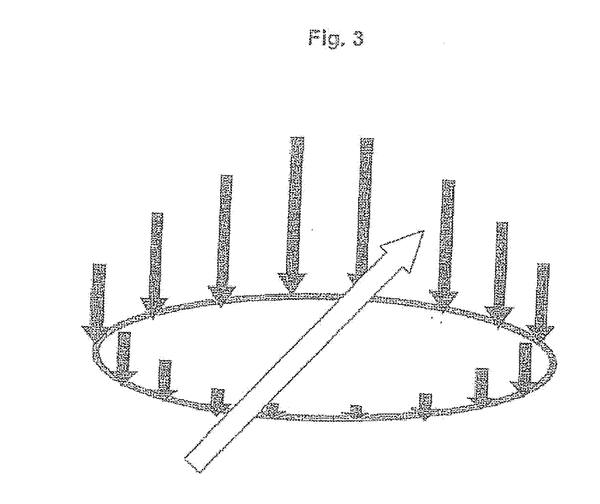

FIG. 3 shows a representation of the pressure applied in the massaging circuit

FIG. 4a shows a device for infrared irradiation (semicircle or segment of a circle)

FIG. 4b shows a device for infrared irradiation (plates)

FIG. 5 shows a diagram of the venular oxygen depletion ΔpO₂ after treatment with first and second device

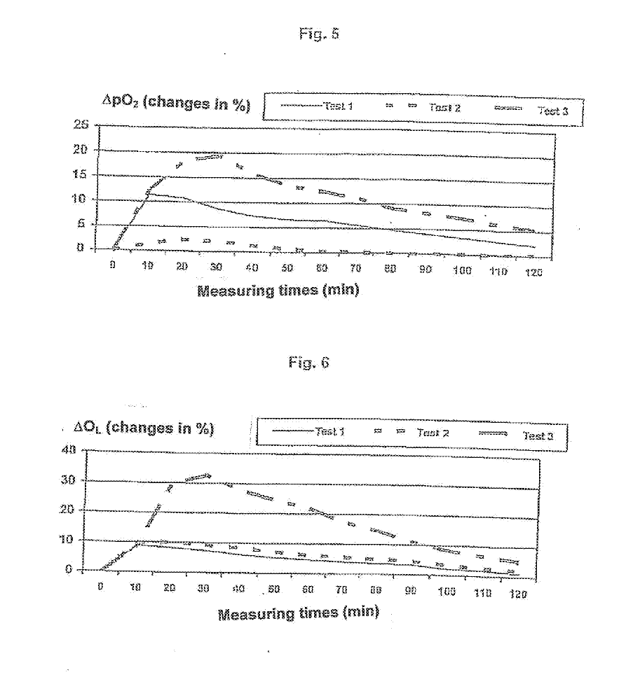

FIG. 6 shows a diagram of the change in the flow rate of the initial lymph ΔQ_{L} after treatment with first and second device

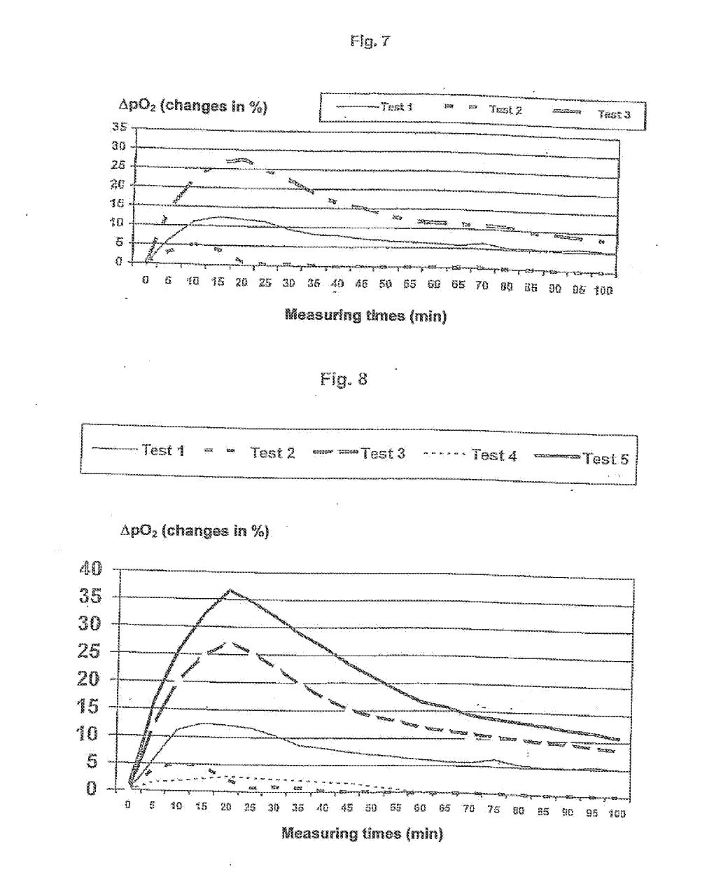

FIG. 7 shows a diagram of the venular oxygen depletion ΔpO₂ after treatment with first and third device

FIG. 8 shows a diagram of the venular oxygen depletion ΔpO₂ after treatment with first, second and third device

In FIG. 1 the individual devices in the apparatus for modulating perfusion of the microcirculation of the blood are represented schematically. The device for generating a pulsed electromagnetic field is represented as mat 1. In this mat, one or more coils are arranged which are connected to a pulse generator (not shown).

Reference numeral 2 denotes the region of action of the device for circulating the lymph flow. The device itself is realized e.g. in the form of a leg cuff which can extend up to the hip of the patient.

Reference numeral 3 also denotes a region, namely the region of action of the device for emitting infrared radiation. The device itself is realized e.g. according to FIG. 4a or FIG. 4b. The three devices are coordinated via a control unit 4, which controls different combinations of the devices among each other and, if applicable, the pulses emitted by each device to the body surface of a patient.

FIG. 2 shows the second device for circulating the lymph flow in the form of a leg cuff. Several massaging arrangements in the form of chambers located horizontally next to each other, of which only the chambers 21 and 22 are illustrated for reasons of simplicity, are driven by the pumps 31 and 32 via a pump controller 35 to be pressurized one after the other with different pressures (increasing and decreasing) according to the predetermined cycle sequence.

After this, the more proximal chambers 23 and 24 (and the further chambers in this circular plane) are pressurized with increasing and decreasing pressures, such that a boost phase and a relaxation phase result in the circular plane.

Preferably, the total pressure increases from the distal to the proximal position, i.e. each circular plane starts at a slightly higher pressure than the previous circular plane.

In FIG. 3 the progression of pressure application in a massaging circuit is represented schematically. The massaging arrangement here consists of 16 chambers, wherein in the left half of the circuit with the chambers 1 to 8 the massaging pressure is increased in said chambers and in the chambers of the right half of the circuit in the chambers 9 to 16 the massaging pressure is designed to be slowly decreasing, as represented by the shortened arrows.

In the further sequence of the chambers according to FIG. 2, all chambers of the subsequent circular plane are driven one after the other in the same manner from the distal to the proximal position, such that the pressure applied from the outside effects a distinct circulation of the lymph flow in the physiological direction of action according to the measured criteria nNP, ΔQven, ΔpO₂, and ΔQ_{L}. These measured data can, therefore, be used for the control of the pressure pulses.

The leg cuff according to the invention encloses the lower limbs and can be controlled with different forces in these two parts, as explained above.

### EXAMPLE 1

With an apparatus according to the invention for modulating perfusion in the microcirculation of the blood, a test series was conducted having combined the first device with the second device, with 42 female probands aged 48 to 57 (without pathological findings) with pronounced orange peel skin phenomenon participating in said test series. 3 subsamples were tested, each comprising 14 probands.
Test 1: Application of the first device (appliance BEMER 3000 plus, manufactured by Innomed AG, Liechtenstein) with groups of pulses of 12 μT lasting 20 seconds, alternating with groups of pulses of 35 μT lasting 0.12 seconds, onto a body-sized mat, whereon the probands were lying in the prone position. Test time 10 minutes; frequency 33 Hz.
Test 2: Application of the second device in the form of two leg cuffs, each comprising 5 massaging circuits arranged above each other. Each circuit contains 7 to 9 chambers. Pressure pulses (air) controlled in 3 phases per minute. The force acting on the skin surface is 2 to 35 N in the relaxation phase and 10 to 65 N in the boost phase; test time 15 minutes.
Test 3: Application of both devices immediately after one another, with a break of 0.5 min. in between.

Measurement of the microcirculation values is performed on the subcutis (thigh, flexor side) at intervals of 10 minutes for an observation time of 120 minutes.

Measurement methods: intravital microscopic examination unit with computer-assisted image evaluation (high-speed camera system), vital microscopic reflection spectrometry, laser DOPPLER microflowmetry/white-light spectroscopy

Investigated characteristics: flow rate of the initial lymph ΔQ_{L}, number of blood cell perfused nodal points in the defined microvascular network nNP, changes in the venular flow rate ΔQven, venular oxygen depletion ΔpO₂. Biometry was performed using the WILCOXON rank-sum test, α=5%.

The results have been given further above. The diagrams in FIG. 5 and FIG. 6 are given as examples of ΔpO₂ and ΔQ_{L}. For test 3, the corresponding contributions at the same measuring times considerably exceed the sum of test 1 and test 2.

### EXAMPLES 2 AND 3

In a manner similar to example 1, test 1 was conducted with the following changes:

### Example 2

Base pulse of the second group of pulses 16 seconds at 22 μT;
supplementary pulse of the second group of pulses 0.15 seconds at 45 μT.

### Example 3

Base pulse of the second group of pulses 18 seconds at 33 μT;
supplementary pulse of the first group of pulses 0.13 seconds at 78 μT.

In both cases comparable results were obtained as in example 1.

### EXAMPLE 4

FIG. 4a shows a preferred variant of the third device for emitting infrared radiation. The device can be designed as a semicircle or as a smaller partial circle. The inner side of the semicircle is equipped with a series of infrared-A diodes, which, in total and with regard to their performance at an average distance of 20 cm from the body surface of a proband, permit an increase in skin temperature of up to 8° C.

With an apparatus according to the invention for modulating perfusion in the microcirculation of the blood, a test series was conducted having combined the first device with the third device, with 36 female probands aged 55 to 62 (without pathological findings) with normal skin type participating in said test series. 3 subsamples were tested, each comprising 12 probands.
Test 1: Application of the first device (appliance BEMER 3000 plus, manufactured by Innomed AG, Liechtenstein) with groups of pulses of 12 μT lasting 20 seconds, alternating with groups of pulses of 44 μT lasting 0.12 seconds onto a body-sized mat, whereon the probands were lying in the prone position. Test time 10 minutes, frequency 33 Hz.
Test 2: Application of the third device with predominantly infrared-A radiation for a period of 10 minutes onto the thigh (flexor side) while lying in the prone position.
Test 3: Application of both devices simultaneously.

Measurement of the microcirculation values is performed on the subcutis (thigh, flexor side) at intervals of 5 minutes.

Measurement methods: intravital microscopic examination unit with computer-assisted image evaluation (high-speed camera system); laser DOPPLER microflowmetry/white-light spectroscopy

Investigated characteristics: number of blood cell perfused nodal points in the defined microvascular network nNP; changes in the venular flow rate ΔQven; venular oxygen depletion ΔpO₂. Biometry was performed using the WILCOXON rank-sum test, α=5%.

The results have been given further above. The diagram in FIG. 7 is given as an example of ΔpO₂. For test 3, the corresponding contributions at the same measuring times considerably exceed the sum of test 1 and test 2

### EXAMPLES 5 AND 6

In a manner similar to example 4, test 1 was conducted with the following changes:

### Example 5

Base pulse of the second group of pulses 22 seconds at 17 μT
supplementary pulse of the first group of pulses 0.12 seconds at 56 μT.

### Example 6

Base pulse of the second group of pulses 19 seconds at 36 μT
supplementary pulse of the first group of pulses 0.10 seconds at 86 μT.

In both cases comparable results were obtained as in example 4.

### EXAMPLE 7

With an apparatus according to the invention for modulating perfusion in the microcirculation of the blood, a test series was conducted having combined the first device with the second and the third device, with 60 female probands aged 55 to 62 (without pathological findings) with normal skin type participating in said test series. 5 subsamples were tested, each comprising 12 probands.
Test 1: Application of the first device (appliance BEMER 3000 plus, manufactured by Innomed AG, Liechtenstein) with groups of pulses of 12 μT lasting 20 seconds, alternating with groups of pulses of 44 μT lasting 0.12 seconds onto a body-sized mat, whereon the probands were lying in the prone position. Test time 10 minutes, frequency 33 Hz.
Test 2: Application of the third device with predominantly infrared-A radiation for a period of 10 minutes onto the thigh (flexor side) while lying in the prone position.
Test 3: Application of both devices simultaneously.
Test 4: Application of the second device in the form of two leg cuffs, each comprising 5 massaging circuits arranged above each other. Each circuit contains 7 to 9 chambers. Pressure pulses (air) controlled in 3 phases per minute. The force acting on the skin surface is 2 to 35 N in the relaxation phase and 10 to 65 N in the boost phase; test time 15 minutes.
Test 5: Application of the first and the third device simultaneously and of the second device immediately following the end of the period of treatment with the first device

Measurement of the microcirculation values is performed on the subcutis (thigh, flexor side) at intervals of 5 minutes.

Measurement methods, investigated characteristics and biometry as in example 4.

The results have been given further above. The diagram in FIG. 8 is given as an example of ΔpO₂. For test 5, the corresponding contributions at the same measuring times considerably exceed the sum of test 3 and test 4.

### EXAMPLE 8

The procedure was as in example 7, wherein in test 5, all three devices simultaneously emitted pulses. ΔpO₂ was at about 40%.

1. Apparatus for modulating the perfusion in the microcirculation of the blood, comprising:
a first device for generating a pulsed electromagnetic field, comprising at least one pulse generator and one magnetic coil, said electromagnetic field having a pulse sequence of at least two synchronous or asynchronous groups of pulses, wherein for the first group of pulses the pulse time is 0.1 to 0.2 seconds at a magnetic flux density of 35 to 100 μT and for the second group of pulses the pulse time is 10 to 30 seconds at a magnetic flux density of 2 to 40 μT; a second device for enhancing the lymph flow, said second device being used to trigger massaging pulses via a massaging arrangement,
wherein the massaging arrangement comprises several circuits of compression chambers, the compression chambers being arranged horizontally next to each other and the circuits being arranged above each other, said circuits being configured such that, via a pulsing device and one or more pumps connected thereto, gradient increasing pressure pulses can be triggered for the compression chambers in one half of the circuit and gradient decreasing pressure pulses can be triggered for the chambers in the other half of the circuit;
a third device for emitting infrared radiation, said third device emitting at least one heat pulse and having an infrared spectrum of 80 to 100% infrared-A, 0 to 19% infrared-B and 0 to 1% infrared-C;
a control unit by means of which the pulses of the three devices are controlled such that either the first and the second device simultaneously emit pulses or the first and the third device simultaneously emit pulses or the first, second and third device simultaneously emit pulses.

2. Apparatus according to claim 1, wherein for the first device the pulse time of a second group of pulses is 10 to 20 seconds at a magnetic flux density of 2 to 40 μT.

3. Apparatus according to claim 1, wherein with the first device, the first and the second group of pulses occur simultaneously in such a manner that the first group of pulses occurs every 10 to 30 seconds two to 6 times per minute and the pulse sequence of the first group of pulses is superimposed on the signal of the second group of pulses.

4. Apparatus according to claim 1, wherein the pulse sequence of the first group of pulses is 10 to 90 μT higher than the pulse sequence of the second group of pulses.

5. Apparatus according to claim 1, wherein the magnetic flux density of the first group of pulses is 40 to 90 μT.

6. Apparatus according to claim 1, wherein the magnetic flux density of the second group of pulses is 5 to 34 μT.

7. Apparatus according to claim 1, wherein the pulse time of the second group of pulses is within the range from 15 to 20 seconds.

8. Apparatus according to claim 1, wherein the first device comprises a pulse generator with a mat connected thereto having flat magnetic coils arranged therein.

9. Apparatus according to claim 1, wherein the control unit emits pulses to the massaging arrangement of the second device in such a manner that compression chambers arranged in series are driven one after the other from the beginning of a row to the end of a row.

10. Apparatus according to claim 9, wherein the massaging arrangement in the second device can be controlled by pressure and has a pressure-controlled build-up of compression, which enables a boost phase and a relaxation phase in the compression chambers.

11. Apparatus according to claim 1, wherein the third device comprises infrared-A radiation of a wavelength of 800 to 1400 nm, contributing to the total infrared radiation with a proportion of at least 90%.

12. Apparatus according to claim 1, wherein for the third device the heat pulse can be triggered as a single sustained pulse over a period of 5 to 30 minutes.

13. Apparatus according to claim 1, wherein the heat pulse of the third device can be controlled via a digitally processed signal of the first device.

14. Massaging device for circulating the lymph flow, wherein in a flat structure, massaging arrangements are arranged horizontally next to each other and vertically in several circular planes above each other, where a pulsing device and a pump controller and one or more pumps connected thereto are used to trigger gradient increasing pressure pulses for the chambers in one half of the circuit and the pumps are used to trigger gradient decreasing pressure pulses for the chambers in the other half of the circuit.

15. Massaging device according to claim 11, wherein the flat structure is a leg cuff having compression chambers as massaging arrangements.

16. Method for modulating perfusion of the micro- and macrocirculation of the blood by external treatment of a human being successively or simultaneously with
a) a pulsed electromagnetic field having a pulse sequence of at least two synchronous or asynchronous groups of pulses, wherein for the first group of pulses the pulse time is 0.1 to 0.2 seconds at a magnetic flux density of 35 to 100 μT and for the second group of pulses the pulse time is 10 to 30 seconds at a magnetic flux density of 2 to 40 μT, and the pulsed electromagnetic field is directed onto at least one part of the human body;
b) massaging pulses via a lymph flow massaging arrangement, where the pulses are emitted via the skin surface onto the tissue of the limbs of a human being and this is done via several circuits of compression chambers, the compression chambers being arranged horizontally next to each other and the circuits being arranged above each other, where gradient increasing pressure pulses are triggered for the compression chambers in one half of the circuit and gradient decreasing pressure pulses are triggered for the chambers in the other half of the circuit and the pressure in each circuit increases from the distal to the proximal position; and
c) infrared irradiation of a part of the human body or the entire human body, where the heat pulse has an infrared spectrum of 80 to 100% infrared-A, 0 to 19% infrared-B and 0 to 1% infrared-C, and where, of the three treatment measures, at least a) and b), a) and c), or a) and b) and c) are applied.

17. Method according to claim 16, wherein the blood microcirculation criteria are determined as a function of the venular oxygen depletion ΔpO₂, the number of blood cell perfused nodal points nNP, the venular flow rate ΔQᵥₑₙ and the flow rate of the initial lymph ΔQ_{L}.

18. Method according to claim 17, wherein the blood macrocirculation criteria are determined as a function of the pressure difference of the right atrium of the heart and the vena cava.

19. Apparatus according to claim 1, wherein the magnetic flux density of the first group of pulses is 30 to 45 μT.

20. Apparatus according to claim 1, wherein the magnetic flux density of the second group of pulses is 9 to 22 μT.
